(19) United States
(12) Patent Application Publication
(10) Pub. No.: US 2026/0224178 A1
(43) Pub. Date: Aug. 6, 2026

(54) POSITRON AND GAMMA-RAY DUAL DETECTOR AND METHOD FOR PROVIDING MALIGNANT TUMOR DIAGNOSIS INFORMATION USING SAME

(71) Applicant: KOREA UNIVERSITY RESEARCH AND BUSINESS FOUNDATION, Seoul (KR)

(72) Inventors: Jung-Yeol YEOM, Seoul (KR); Hyun Koo KIM, Seoul (KR); Chanho KIM, Seoul (KR); Semin KIM, Seoul (KR); Dongyoung KIM, Suwon-si (KR); Yeeun LEE, Seoul (KR)

(73) Assignee: KOREA UNIVERSITY RESEARCH AND BUSINESS FOUNDATION, Seoul (KR)

(21) Appl. No.: 18/881,397

(22) PCT Filed: Jun. 26, 2023
(86) PCT No.: PCT/KR2023/008805

§ 371 Date: Oct. 7, 2025

(30) Foreign Application Priority Data
Jul. 6, 2022 (KR) 10-2022-0083191

(51) Int. Cl. A61B 6/42; A61B 6/00; A61B 6/12

(52) U.S. Cl. CPC A61B 6/4258; A61B 6/12; A61B 6/5217; A61B 2560/0252

(57) ABSTRACT

The present invention relates to a positron and gamma-ray dual detector and a method for providing malignant tumor diagnosis information by using same. The positron and gamma-ray dual detector according to an embodiment of the present invention comprises: a scintillator unit (10) that includes a first scintillator (11) which emits a first flash by reaction with positrons, and a second scintillator (13) which is arranged adjacent to the first scintillator (11) and emits a second flash by reaction with gamma rays having passed through the first scintillator (11); an photosensor (20) that senses at least one of the first flash and the second flash and generates and outputs a sensed signal; a casing (30) that accommodates the scintillator unit (10) and the photosensor (20) therein and has, at one end, an inlet hole (31) that allows positrons or gamma rays to enter the first scintillator (11); and a signal processor (40) that receives the output sensed signal and identifies, on the basis of the received sensed signal, positrons and gamma rays entered through the inlet hole (31).

### TECHNICAL FIELD

The present invention relates to a positron/gamma-ray dual detector and a method for providing information for diagnosing a malignant tumor using the same, and more specifically, to a device for distinguishing and detecting positrons and gamma rays, and a method for providing information capable of identifying a malignant tumor using a new cancer detection index and the device.

### BACKGROUND ART

In Korea, not only is the population aging rapidly, but the incidence of cancer is also rapidly increasing every year. In Korea, except for relatively mild thyroid cancer, the incidence of stomach cancer and colorectal cancer is the highest, and Korea has the highest incidence of stomach cancer and colorectal cancer in the world (World Cancer Research Foundation). Accordingly, rather than uniformly applying the existing surgical method of widely resecting the normal area including the cancer, a precise, patient-tailored minimally invasive cancer surgery of minimally resecting the normal area including the cancer is needed to minimize post-treatment complications and improve the patient's quality of life.

Nuclear medicine imaging equipment using radioisotopes can visualize functional/biochemical information of a living body, and thus is useful for identifying pathological phenomena, diagnosing diseases, determining prognosis after treatment, and planning treatments. In addition, unlike visible light-, ultrasound-, and X-ray-based techniques, nuclear medicine techniques provide functional information (e.g., whether a tumor is malignant) rather than structural information, and thus are often used in hospitals to diagnose cancer, etc. Accordingly, many devices in the form of portable nuclear medicine detectors have been used for intraoperative cancer detection or sentinel lymph node biopsy.

Currently, radio-guided surgery mainly involves administering a radiopharmaceutical for nuclear medicine imaging to a patient and then measuring the radiation emitted from the radiopharmaceutical with a detector. When ¹⁸F-FDG (fluorodeoxyglucose) labeled with a radioisotope (¹⁸F) that emits positrons is administered to a patient, the patient's body recognizes FDG as glucose, and the major tissues where glycolysis occurs absorb FDG. Most cancer cells have more active glycolysis than normal cells, and thus absorb large amounts of FDG. Over time, the radioisotope of FDG decays, emitting positrons, which then annihilate with electrons (positron-electron annihilation reaction) to produce two gamma rays. Therefore, by measuring positrons and gamma rays using a nuclear medicine detector, it is possible to determine the presence or absence of cancer.

The nuclear medicine detectors that are commonly used are generally gamma-ray detectors and positron detectors. Gamma-ray detectors can identify the location of cancer without dissection by utilizing the characteristics of highly penetrable gamma rays, but there are cases where it is difficult to accurately identify sentinel lymph nodes or cancer, due to the shine-through effect caused by gamma rays generated around lesion tissue. To overcome this difficulty, a positron detector is used. Since the mean free path length of positrons in the body is very short (within a few mm), the positron detector can accurately detect the location of a tumor, and can also detect even relatively small tumors.

Conventional cancer detection methods using nuclear medicine detectors use the count of positrons or gamma rays measured in cancer and surrounding tissues using positron detectors or gamma-ray detectors that detect only positrons or gamma rays. Since the travel distance of positrons in tissues is very short, positron detectors can accurately detect even small-sized tumor tissues. In addition, since gamma-ray detectors detect gamma rays with strong penetrability, they can be used to primarily localize tumor tissue, instead of positron detectors, which have a very short detection range. Due to the high glucose metabolism of malignant tumors, radiopharmaceuticals such as ¹⁸F-FDG often exhibit tumor-to-background ratios (TBRs) exceeding 10:1. Accordingly, positron detectors and gamma-ray detectors generally identify and localize malignant tumors based on the TBR value. However, TBR is a background-dependent index, and the TBR value changes depending on the radiation intensity of the selected background. For example, if a malignant tumor is located in a site with a high gamma-ray signal, such as the kidney, heart, or bladder, gamma rays in the background are measured as positrons, and thus false positron signals increase, making it difficult to identify the tumor using the TBR value.

Therefore, there is an urgent need for a solution to the problems of conventional nuclear medicine detectors and cancer detection methods based on TBR values.

### DISCLOSURE

### Technical Problem

The present invention has been made in order to solve the above-described problems occurring in the prior art, and one aspect of the present invention is to provide a nuclear medicine positron/gamma-ray dual detector capable of simultaneously measuring positrons and gamma rays, and a method for providing information for diagnosing a malignant tumor, which is capable of detecting a malignant tumor by establishing a new cancer detection index, which is not dependent on the location of the malignant tumor, by using the dual detector.

### Technical Solution

A positron/gamma-ray dual detector according to an embodiment of the present invention includes: a scintillator unit including a first scintillator configured to react with positrons and emit a first scintillation, and a second scintillator disposed adjacent to the first scintillator and configured to react with gamma rays transmitted through the first scintillator and emit a second scintillation; a photosensor configured to detect any one or more of the first scintillation and the second scintillation and generate and output a detection signal; a casing configured to accommodate therein the scintillator unit and the photosensor and having an inlet hole formed at one end to allow the positrons or the gamma rays to enter the first scintillator; and a signal processing unit configured to receive the output detection signal and identify the positrons and gamma rays, which entered through the inlet hole, based on the received detection signal.

In addition, the positron/gamma-ray dual detector according to an embodiment of the present invention may further include a collimator which is detachably attached to one end of the casing and has a gamma-ray passage that communicates with the inlet hole of the casing so that the gamma rays emitted in a predetermined direction are incident on the first scintillator.

In addition, in the positron/gamma-ray dual detector according to an embodiment of the present invention, one end of the casing may be inserted into and screwed to the collimator, and the dual detector may further include a ring-shaped cover sleeve that is screwed to one end of the casing after the collimator is detached.

In addition, in the positron/gamma-ray dual detector according to an embodiment of the present invention, the first scintillator may include any one or more selected from the group consisting of CaF₂(Eu), plastic, and stilbene.

In addition, in the positron/gamma-ray dual detector according to an embodiment of the present invention, the second scintillator may include any one or more selected from the group consisting of GSO, GPS, LuAG, GAGG, BGO, YAP, and LYSO.

In addition, the positron/gamma-ray dual detector according to an embodiment of the present invention may further include a temperature sensor that detects the temperature of the photosensor and generates and outputs temperature information, and the signal processing unit may receive the output temperature information and compensate for the detection signal.

In addition, the positron/gamma-ray dual detector according to an embodiment of the present invention may further include: a handle connected to the other end of the casing and formed to be gripped by a user; and a signal cable that penetrates the inside of the handle and transmits the detection signal to the signal processing unit.

In addition, in the positron/gamma-ray dual detector according to an embodiment of the present invention, the signal control unit may identify the positrons and the gamma rays based on the waveform of the detection signal.

Meanwhile, a method for providing information for diagnosing a malignant tumor according to an embodiment of the present invention includes: step (a) of sequentially placing the positron/gamma-ray dual detector on multiple tissue regions of a subject's body, identifying positrons and gamma-rays emitted from each of the tissue regions, and counting the positrons and the gamma-rays; step (b) of calculating a cancer index value for each of the tissue regions according to the following Equation 1; and step (c) of comparing the cancer index value calculated for each of the tissue regions with a predetermined critical value and determining that the tissue region corresponding to the cancer index value exceeding the critical value is a malignant tumor tissue:

$$PGR = \frac{{Counts}_{β}}{{Counts}_{γ - ray}} \qquad [ Equation 1 ]$$

wherein PGR is the cancer index value, Counts_{β} is the positron count, and Counts_{γ-ray} is the gamma-ray count.

In addition, in the method for providing information for diagnosing a malignant tumor according to an embodiment of the present invention, the critical value may be the average value of the cancer index values calculated for each of the tissue regions plus n times the standard deviation (where n is a natural number greater than or equal to 1).

In addition, in the method for providing information for diagnosing a malignant tumor according to an embodiment of the present invention, n may be 3.

The features and advantages of the present invention will become more apparent from the following detailed description taken with reference to the accompanying drawings.

The terms or words used in the specifications and claims should not be construed as usual or dictionary definition but should be rather construed to be consistent with the technical spirits of the present invention based on the principle that the inventor may properly define the terms to describe his/her invention in the best manner.

### Advantageous Effects

According to the present invention, it is possible to enable more accurate diagnosis by acquiring functional/biochemical information in real time using a nuclear medicine positron/gamma-ray dual detector. In addition, it is expected that it will be possible to determine the extent of tissue resection according to the individual's disease condition, thereby providing patient-tailored surgery and contributing to overcoming cancer. Furthermore, it is possible to provide improved surgical outcomes through patient-tailored minimally invasive cancer surgery, thereby minimizing complications and after-effects, thus improving the quality of life of patients and contributing to the promotion of national health.

### BRIEF DESCRIPTION OF DRAWINGS

FIG. 1 is a perspective view of a positron/gamma-ray dual detector according to an embodiment of the present invention.

FIG. 2 is a cross-sectional view of the positron/gamma-ray dual detector according to an embodiment of the present invention.

FIG. 3 shows a method for identifying positrons and gamma-rays using the positron/gamma-ray dual detector according to an embodiment of the present invention.

FIG. 4 is a cross-sectional view of a positron/gamma-ray dual detector according to another embodiment of the present invention.

FIG. 5 illustrates the coupling relationship between a casing and a collimator in the positron/gamma-ray dual detector according to another embodiment of the present invention.

FIG. 6 is a cross-sectional view of the positron/gamma-ray dual detector according to another embodiment of the present invention.

FIG. 7 is a flow diagram showing a method for providing information for diagnosing a malignant tumor according to an embodiment of the present invention.

FIG. 8 is a graph showing the signal of a CaF₂(Eu) plus GSO dual scintillator according to Experimental Example 1.

FIG. 9 is graphs showing the distributions of PSD parameters for gamma-ray signals (a) and positron/gamma-ray signals (b) of a CaF₂(Eu)/GSO dual scintillator according to Experimental Example 1.

FIG. 10 is a diagram showing the configuration of a positron/gamma-ray dual detection system according to Experimental Example 1.

FIG. 11 schematically shows a probe of a positron/gamma-ray dual detection system according to Experimental Example 1.

FIG. 12 is photographs showing the gamma-ray mode (a) and the positron mode (b) of the probe shown in FIG. 11.

FIG. 13 shows the PCB and PCB layout of the front-end circuit shown in FIG. 10.

FIG. 14 is a flow block diagram of the digital circuit shown in FIG. 10.

FIG. 15 illustrates an experiment for measuring the performance of the gamma-ray mode and positron mode of the probe.

FIG. 16 shows PET/CT images of skin cancer in rabbits used in an animal experiment according to Experimental Example 1.

FIG. 17 shows PSF (Point Spread Function) graphs of a positron/gamma-ray dual detection probe and a commercial probe according to Experimental Example 1.

FIG. 18 shows TBR values measured according to the gamma-ray mode (a) and positron mode (b) of a positron/gamma-ray dual detection probe in an animal experiment according to Experimental Example 1.

FIG. 19 illustrates a conventional positron count-based malignant tumor detection method.

FIG. 20 illustrates an in vitro test according to Experimental Example 2.

FIG. 21 illustrates an in vivo test according to Experimental Example 2.

FIG. 22 is graphs showing the changes in positron count (a) and PGR value (b) as a function of the distance between a ²²Na gamma-ray source and a positron/gamma-ray dual detection probe in an in vitro test according to Experimental Example 2.

FIG. 23 is graphs showing the changes in positron count (a) and PGR value (b) as a function of the distance between a positron/gamma-ray dual detection probe in close contact with a positron source and a gamma-ray source in an in vitro test.

FIG. 24 shows PET/CT images of rabbit tumors taken before an in vivo test according to Experimental Example 2.

FIG. 25 shows the results of an in vivo test according to Experimental Example 2.

FIG. 26 is graphs showing the positron count and PGR value of residual cancer according to Experimental Example 2.

### BEST MODE

The objects, specific advantages, and novel features of the present invention will become more apparent from the following detailed description and preferred embodiments taken in conjunction with the accompanying drawings. It should be noted that, when adding reference numerals to components in each drawing in the present invention, identical components are given the same reference numerals as much as possible even if they are shown in different drawings. In addition, terms such as “first,” “second,” etc. are used to distinguish one component from another component, and the components are not limited by these terms. In the following description of the present invention, a detailed description of related known technologies will be omitted if it may unnecessarily obscure the subject matter of the present invention.

Hereinafter, preferred embodiments of the present invention will be described in detail with reference to the accompanying drawings.

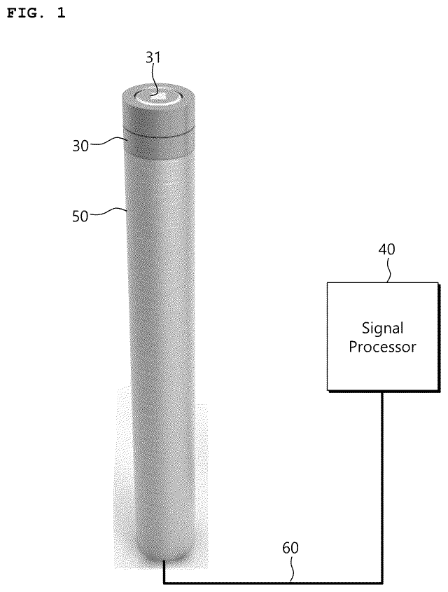

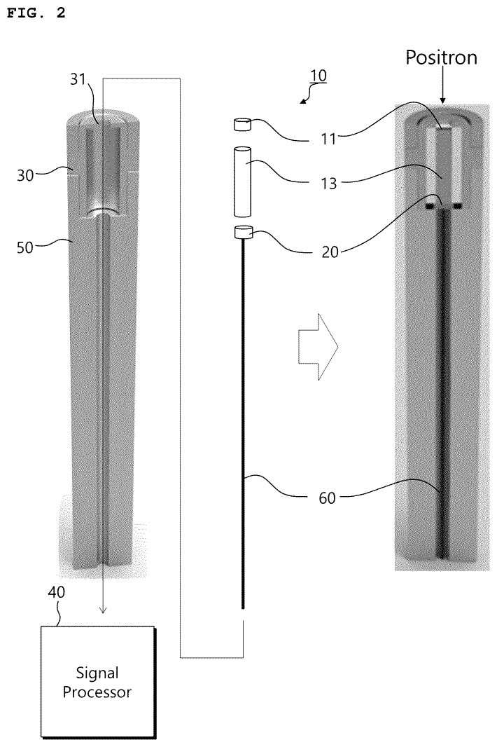

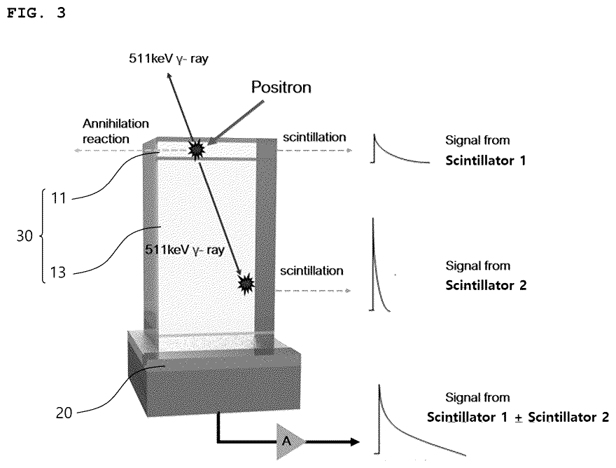

FIG. 1 is a perspective view of a positron/gamma-ray dual detector according to an embodiment of the present invention, FIG. 2 is a cross-sectional view of the positron/gamma-ray dual detector according to an embodiment of the present invention, and FIG. 3 shows a method for identifying positrons and gamma-rays in a positron/gamma-ray dual detector according to an embodiment of the present invention.

As shown in FIGS. 1 to 3, a positron/gamma-ray dual detector according to an embodiment of the present invention includes: a scintillator unit 10 including a first scintillator 11 configured to react with positrons and emit a first scintillation, and a second scintillator 13 disposed adjacent to the first scintillator 11 and configured to react with gamma rays transmitted through the first scintillator 11 and emit a second scintillation; a photosensor 20 configured to detect any one or more of the first scintillation and the second scintillation and generate and output a detection signal; a casing 30 configured to accommodate therein the scintillator unit 10 and the photosensor 20 and having an inlet hole 31 formed at one end to allow positrons or gamma rays to enter the first scintillator 11; and a signal processing unit 40 configured to receive the output detection signal and identify positrons and gamma rays, which entered through the inlet hole 31, based on the received detection signal.

The present invention relates to a positron/gamma-ray dual detector capable of distinguishing and detecting both positrons and gamma-rays, and a method for providing information for diagnosing a malignant tumor, which is capable of proposing a new cancer detection index, which is not dependent on the location of the malignant tumor, by using the dual detector, and determining the presence or absence of a malignant tumor based on the cancer detection index.

First, a positron/gamma-ray dual detector according to an embodiment of the present invention will be described. Specifically, the positron/gamma-ray dual detector according to an embodiment of the present invention includes a scintillator unit 10, a photosensor 20, a casing 30, and a signal processing unit 40.

The scintillator unit 10 is composed of a dual scintillator structure in which a first scintillator 11 and a second scintillator 13 are stacked and disposed adjacent to each other. Here, the first scintillator 11 is a scintillator that reacts with positrons and emits a first scintillation. In addition, the first scintillator 11 consists of a scintillator that has a low density and thus a low probability of interacting with gamma rays. The first scintillator 11 may include any one or more selected from the group consisting of CaF₂(Eu), plastic, and stilbene. However, the first scintillator 11 is not necessarily limited thereto, and may include any material that may react with positrons and emit scintillation.

The second scintillator 13 is a scintillator that reacts with gamma rays and emits a second scintillation, and has scintillation characteristics different from those of the first scintillator 11. Positrons and gamma rays are incident through one end of the scintillator unit 10, and the first scintillator 11 is disposed at the one end. Accordingly, the incident positrons and gamma rays are incident on the first scintillator 11. Here, when positrons are incident, the positrons mostly react with the first scintillator 11 because they have has a short range. At this time, pair annihilation with electrons occurs, producing two 511 keV gamma rays, one of which passes through the first scintillator 11 and reacts with the second scintillator 13, and the other escapes to the outside. Therefore, when positrons are incident, the scintillator unit 10 emits a first scintillation and a second scintillation (see FIG. 3). On the other hand, when gamma rays are incident, the gamma rays pass through the first scintillator 11 and react with the second scintillator 13 to emit the second scintillation, because they have a long range. For example, the second scintillator 13 may include any one or more selected from the group consisting of GSO, GPS, LuAG, GAGG, BGO, YAP, and LYSO.

The photosensor 20 is an element that detects the scintillation emitted from the scintillator unit 10, converts the same into an electrical signal, and outputs the electrical signal. This photosensor 20 is disposed to face the second scintillator 13. Therefore, when positrons and/or gamma rays are incident on the scintillator unit 10, the photosensor 20 detects at least one of the first scintillation and the second scintillation, and generates and outputs a detection signal. As this photosensor 20, a photodiode, an avalanche photodiode (APD), a silicon photomultiplier (SiPM), a photomultiplier tube (PMT), etc. may be used.

The casing 30 is a member that accommodates the scintillator unit 10 and the photosensor 20 therein. An inlet hole 31 is formed at one end of the casing 30 so as to communicate with the internal space, and the first scintillator 11 is disposed closest to the inlet hole 31, and is followed by the second scintillator 13 and the photosensor 20 in that order. Accordingly, positrons or gamma rays pass through the inlet hole 31, enter the interior of the casing 30, and are incident on the first scintillator 11.

The signal processing unit 40 receives the detection signal output from the photosensor 20 and identifies positrons and gamma rays based on the received detection signal. The signal processing unit 40 is composed of electrical and electronic components, circuits, etc. and processes the detection signal. The signal processing unit 40 may perform calculations according to a predetermined algorithm and may include a counter capable of counting positrons and gamma rays.

The signal processing unit 40 may identify positrons and gamma rays based on the waveform of the detection signal. For example, a pulse shape discrimination (PSD) algorithm may be used to distinguish the type of incident radiation. PSD is a technology used to distinguish between different types of radiation (gamma rays, alpha (α) particles, beta (β) particles, and neutrons). Incident radiations react with a detector and generate different types of signals depending on the energy, mass, and charge of the incident particles, and the type of radiation is distinguished by using the difference between these signals. Examples of PSD methods include analog PSD methods such as zero crossing method, charge comparison method, and rise time discrimination methods, and digital PSD methods such as pulse gradient analysis, fuzzy logic, and discrete wavelet transform methods. In addition, various methods capable of distinguishing wavelength discrimination signals may be used. Since positrons produce gamma rays by annihilation with electrons, distinguishing between positrons and gamma rays is essential for measuring positrons. Referring to FIG. 3, when a positron is incident on the scintillator unit 10, the positron reacts with the first scintillator 11 and emits a first scintillation, and a gamma ray produced by the positron-electron annihilation reaction reacts with the second scintillator 13 and emits a second scintillation, so that a signal corresponding to the sum of the first signal according to the first scintillator 11 and the second signal according to the second scintillator 13 is generated. Since the gamma ray passes through the first scintillator 11, and then reacts with the second scintillator 13 and emits a second scintillation, no signal is generated by the first scintillator 11. Since the signal of the first scintillator 11 has a very long decay time, it has a relatively low height and a long signal tail compared to the signal of the second scintillator 13. Since the shapes of the signals are different in this way, the incident positron and gamma ray may be distinguished from each other by comparing the waveforms of the signals.

The signal processing unit 40 does not necessarily have to distinguish between positrons and gamma rays only by pulse waveform discrimination. As another example, positrons and gamma rays may be distinguished from each other based on deep learning technology.

Meanwhile, the positron/gamma-ray dual detector according to an embodiment of the present invention may further include a handle 50 and a signal cable 60.

The handle 50 is connected to the other end of the casing 30 and is formed to be gripped by a user. The signal cable 60 is provided to transmit a detection signal generated by the photosensor 20 to the signal processing unit 40, and it penetrates the inside of the handle 50 and electrically connects the photosensor 20 to the signal processing unit 40.

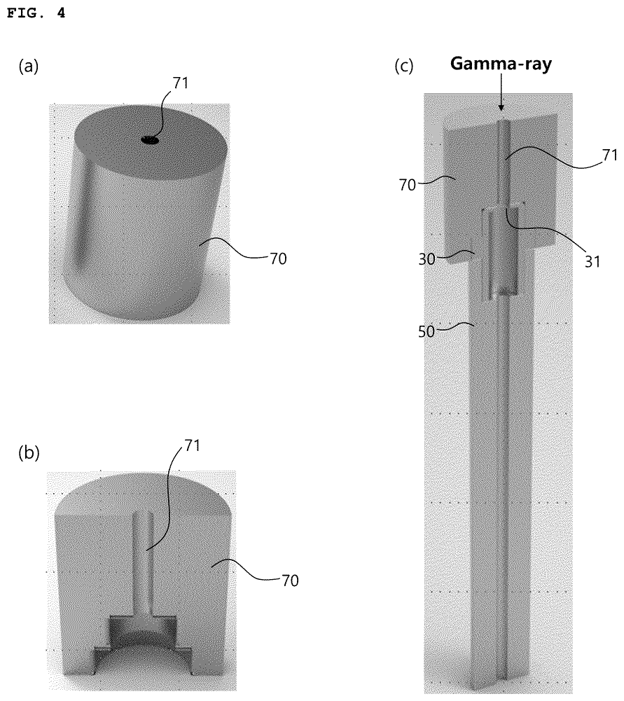

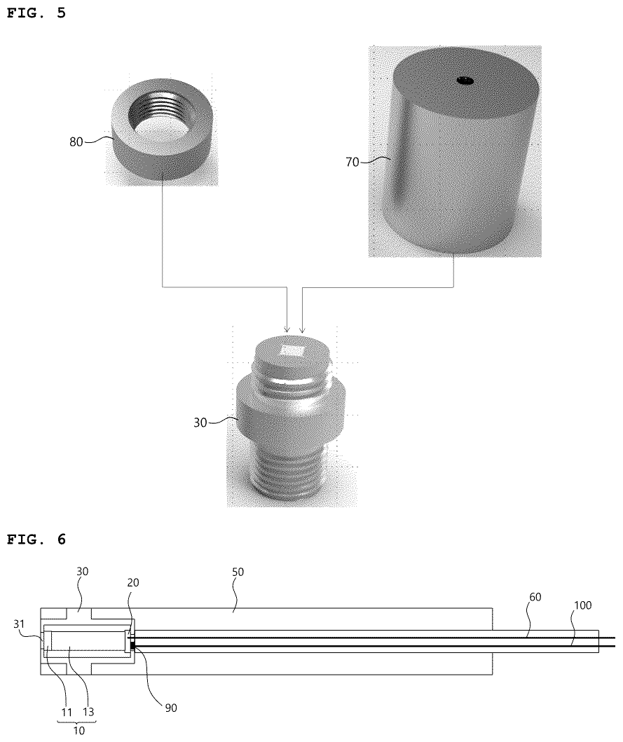

FIG. 4 is a cross-sectional view of a positron/gamma-ray dual detector according to another embodiment of the present invention, and FIG. 5 illustrates the coupling relationship between a casing and a collimator in the positron/gamma-ray dual detector according to another embodiment of the present invention.

As shown in FIGS. 4 and 5, the positron/gamma-ray dual detector according to another embodiment of the present invention may further include a collimator 70.

The collimator 70 is a member formed in a tubular shape, is detachably attached to one end of the casing 30, and has a gamma-ray passage 71 that communicates with the inlet hole 31 of the casing 30. This collimator 70 selectively causes only gamma rays emitted in a predetermined direction to be incident on the first scintillator 11.

The positron/gamma-ray dual detector according to another embodiment of the present invention may be used in two modes depending on whether the collimator 70 is detached or not. In the gamma-ray detection mode for measuring gamma-rays, the collimator 70 capable of limiting the direction of gamma-rays is required in order to accurately detect a desired region, because gamma-rays have high penetrability. The collimator 70 may be made of a high-density material such as lead, tungsten, etc. in order to attenuate gamma rays emitted in an undesired direction. In the positron detection mode for measuring positrons, the collimator 70 used in gamma-ray measurement is removed and only the detector prototype is used. Therefore, when measuring gamma rays, the collimator 70 is used in a state attached to one end of the casing 30, and when measuring positrons, the dual detector is used after the collimator 70 is detached.

The detachable attachment of the collimator 70 to the casing 30 may be done by screwing. In this case, screw threads may be formed on the exterior of one end of the casing 30 and the inner wall of the end of the collimator 70 so that one end of the casing 30 may be screwed while being inserted into the end of the collimator 70. Meanwhile, in a state in which the collimator 70 had been removed, a cover sleeve 80 may be attached to one end of the casing 30. The cover sleeve 80 may be formed in a ring shape having a hollow space in the center, so that one end of the casing 30 may be screwed while being inserted into the hollow space.

FIG. 6 is a cross-sectional view of the positron/gamma-ray dual detector according to another embodiment of the present invention.

Referring to FIG. 6, the positron/gamma-ray dual detector according to another embodiment of the present invention may further include a temperature sensor 90. Here, the temperature sensor 90 is disposed in proximity to the photosensor 20 to detect the temperature of the photosensor 20 and generate and output temperature information. The output temperature information may be transmitted to the signal processing unit 40 through a separate cable 100 other than the signal cable 60 described above. While the photosensor 20 converts the scintillation into an electrical signal and outputs the same, a temperature change in the photosensor 20 occurs, and thus an error may occur in the gain of the photosensor 20 depending on the temperature change. Accordingly, the temperature of the photosensor may be measured through the temperature sensor 90, and the signal processing unit 40 may compensate for the detection signal based on the temperature information. An example of the temperature sensor 90 may be a thermistor. However, the temperature sensor 90 is not necessarily limited thereto.

Hereinafter, a method for providing information for diagnosing a malignant tumor according to an embodiment of the present invention will be described. Since the method for providing information for diagnosing a malignant tumor according to the present invention is performed using the above-described positron/gamma-ray dual detector which is as described above, contents overlapping with those of the positron/gamma-ray dual detector will be omitted or only briefly described.

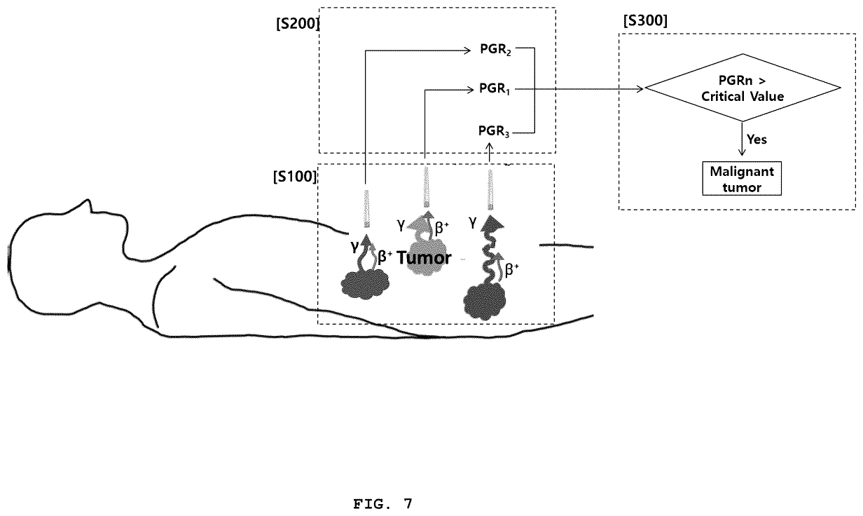

FIG. 7 is a flow diagram showing a method for providing information for diagnosing a malignant tumor according to an embodiment of the present invention.

As shown in FIG. 7, the method for providing information for diagnosing a malignant tumor according to an embodiment of the present invention includes: step (S100) of sequentially positioning the positron/gamma-ray dual detector in multiple tissue regions of a subject's body, identifying positrons and gamma-rays emitted from each of the tissue regions, and counting the positrons and the gamma-rays; step (S200) of calculating a cancer index value for each of the tissue regions according to the following Equation 1; and step (S300) of comparing the cancer index value calculated for each of the tissue regions with a predetermined critical value and determining that a tissue region corresponding to the cancer index value exceeding the critical value is a malignant tumor tissue:

$$PGR = \frac{{Counts}_{β}}{{Counts}_{γ - ray}} \qquad [ Equation 1 ]$$

wherein PGR is the cancer index value, Counts_{β} is the positron count, and Counts_{γ-ray} is the gamma-ray count.

In a conventional art, an index called tumor-to-background ratio (TBR), which is the ratio of positron counts measured in a tumor and the background near the tumor, was used to determine malignancy. Empirically, if the TBR value is 1.5 or higher, it is determined to be cancer. However, some gamma rays can be misdetected as positrons due to the positron detection principle, and thus if a tumor is located around organs that have high FDG uptake and emit a large amount of gamma rays, such as the liver, heart, and kidneys, it is difficult to accurately determine malignancy using TBR, a background-dependent index. Since this can create a situation where normal tissue is unnecessarily resected during cancer resection surgery or residual cancer exists, a new cancer detection index is needed to minimize postoperative complications and after-effects for patients. Accordingly, the present invention proposes a new cancer detection index that may be determined using the above-described positron/gamma-ray dual detector, and has devised a method capable of diagnosing a malignant tumor using the same.

Specifically, the method for providing information for diagnosing a malignant tumor according to an embodiment of the present invention includes a positron/gamma-ray detection step (S100), a cancer index value calculation step (S200), and a malignant tumor tissue determination step (S300).

In the positron/gamma-ray detection step (S100), a radiopharmaceutical such as ¹⁸F-FDG (fluorodeoxyglucose) is administered, and the positron/gamma-ray dual detector are placed on multiple tissue regions of a subject's body after skin incision to detect positrons and gamma-rays emitted from each of the tissue regions. Here, the positron/gamma-ray dual detector identifies positrons and gamma-rays and counts positrons and gamma-rays.

In the cancer index value calculation step (S200), cancer index values (PGR₁, PGR₂, PGR₃, PGRₙ) are calculated for each of the tissue regions, which is the target of positron and gamma ray detection, according to the following Equation 1:

$$PGR = \frac{{Counts}_{β}}{{Counts}_{γ - ray}} \qquad [ Equation 1 ]$$

wherein PGR is the cancer index value, Counts_{β} is the positron count, and Counts_{γ-ray} is the gamma-ray count.

Positron count-to-Gamma count Ratio (PGR) is a new cancer detection index proposed in the present invention to replace the conventional TBR. Since a certain percentage of gamma rays incident on the positron/gamma-ray dual detector are detected as a positron signal, the positron count-to-gamma count ratio (PGR) due to background gamma-rays is constant. However, in a malignant tumor, the emitted positrons are directly detected in addition to the false positron signal generated by background gamma rays, and thus the PGR value is always higher than the background regardless of the location of the malignant tumor.

The malignant tumor tissue determination step (S300) determines, based on the cancer index value calculated for each tissue region, whether the tissue is a malignant tumor tissue. Specifically, the cancer index value for each tissue region is compared with a predetermined critical value, and a tissue region exceeding the critical value is determined to be a malignant tumor tissue.

Here, the critical value may be defined as the average value of the cancer index values calculated for each tissue region plus n times the standard deviation (where n is a natural number greater than or equal to 1). For example, the average value of the cancer index values calculated for each tissue region plus three times the standard deviation is set as the critical value, the cancer index values are compared with the critical value, and it is determined that the corresponding tissue region having the cancer index value exceeding the critical value is a malignant tumor tissue. That is, if the PGR value measured in the cancer tissue is greater than the average value of the PGR values measured in several surrounding normal tissues (background) plus three times the standard deviation, it is determined to be cancer. The probability that general data exists outside ±3σ according to the 3-sigma rule is 0.3%, and thus, in this case, a relatively strict standard is used for determining the presence or absence of cancer.

PGR can accurately distinguish between cancer tissue and normal tissue because the positron count-to-gamma count ratio is constant even when a large amount of positrons are detected due to an organ where glycolysis is active. In this way, PGR has a stricter standard than the existing index, TBR, and is not greatly affected by the surrounding normal tissue, i.e., the background, and it can solve the problem that discrimination errors may occur in cancer near an organ where glycolysis is active.

[Description of Reference Numerals]

| | |
|---|---|
| 10: scintillator unit | 11: first scintillator |
| 13: second scintillator | 20: photosensor |
| 30: casing | 31: inlet hole |
| 40: signal processing unit | 50: handle |
| 60: signal cable | 70: collimator |
| 80: cover sleeve | 90: temperature sensor |

### MODE FOR INVENTION

Hereinafter, the present invention will be described in more detail by way of experimental examples.

### 1. Experimental Example 1: PSD-Based Positron/Gamma-Ray Dual Detection Probe

### 1.1 Introduction

Since positrons have very limited tissue penetration ability (a few millimeters), positron probes are less likely to experience shine-through by positrons emitted from background tissues with high FDG uptake. However, since many annihilation gamma rays are generated by bremsstrahlung of positrons and positron-electron annihilation reactions within the tissue, it is essential to distinguish positrons from background gamma rays in order to detect positrons. However, due to the similar energy levels of positrons and gamma rays generated by positrons, it is difficult to distinguish between positrons and background gamma rays based on energy alone.

PSD (Pulse Shape Discrimination), a technology that distinguishes the types of radiation particles, distinguishes the types of radiation particles by taking into account the fact that pulse signals exhibit slightly different shapes depending on the type of radiation particle. However, unlike other radiation particles, positrons and gamma rays leave fundamentally the same pulse shape within a scintillator, and thus it is not easy to distinguish particles within a scintillator based solely on the shape of the pulse. As a method of solving this problem, a detector is designed by stacking two layers of scintillators with different characteristics, and the difference in the range between positrons and gamma rays can be used to distinguish and detect positrons. Also, since this type of detector can detect gamma rays differentially from positrons, it is also possible to detect both positrons and gamma rays. In conclusion, a detector with a dual scintillator structure can serve as both a positron probe and a gamma-ray probe.

The main application areas where positron probes and high-energy gamma-ray probes are used are the same, metabolic targeted resections, which are represented by cancer resections, but the roles of the two types of probes during surgery are different. Since the high-energy gamma-ray probes detect gamma rays with strong penetrating ability, they can primarily localize tumor tissue without resection of the lesion site. However, the high-energy gamma-ray probes have a considerably large diameter because a thicker collimator is needed to shield gamma rays with strong penetrating ability. On the other hand, the positron probes can accurately localize tumor tissue with minimal collimation because it is not needed to shield positrons coming from the background, due to the short range of positrons. Due to the small diameter of these positron probes (10 to 15 mm), the positron probes are potentially applicable to minimally invasive surgery. However, since the positron probes require surface detection, resection of the lesion site is necessary for tumor localization.

Therefore, if it is possible to detect both positrons and high-energy gamma rays with one probe, it is possible to accurately localize not only primary tumors but also small residual cancer before lesion resection, thereby completely resecting the cancer. In addition, since both probes equally track positron radiopharmaceuticals such as ¹⁸F-FDG, it is possible to easily detect both positrons and high-energy gamma rays with one probe.

In the following experimental examples, a positron/gamma-ray dual detection probe capable of detecting both positrons and gamma rays was fabricated, and PSD technology was applied thereto to distinguish and detect positrons and gamma rays. When measuring gamma rays, a gamma-ray collimator was mounted to shield gamma rays generated from the side (hereinafter referred to as “gamma-ray mode”). When measuring positrons, there is no need to mount the collimator (hereinafter referred to as “positron mode”). The performance of the fabricated positron/gamma-ray dual detection probe was tested in terms of sensitivity and spatial resolution using a ²²Na positron source and a ²²Na gamma-ray source, and the cancer detection ability thereof was evaluated using a medium-sized animal (rabbit) to which a skin cancer model was applied.

### 1.2 PSD-Based Positron/Gamma-Ray Dual Detection Algorithm

PSD has been used in various fields to distinguish radiation particles. The main interest of PSD is to distinguish gamma rays and neutrons. Gamma rays and neutrons generate secondary particles, recoil electrons and recoil protons, respectively, through reactions such as Compton scattering, photoelectric absorption, or neutron elastic scattering within a scintillator. These secondary particles have different energy loss rates within the scintillator, and have different fractions of delayed fluorescence depending on this difference in energy loss rates. Consequently, the signals generated within the scintillator by gamma rays and neutrons have waveforms with different fractions of delayed fluorescence. Since the fraction of delayed fluorescence signals generated by neutrons is generally higher, the tail of the neutron signal shows a more delayed attenuation than the gamma-ray signal, and thus gamma-rays and neutrons can be distinguished from each other using PSD. However, unlike the signals of gamma rays and neutrons, which have different waveforms within a scintillator, recoiled electrons, which are secondary particles generated by gamma rays, have all properties (except charge) identical to positrons due to the matter-antimatter relationship. Therefore, gamma rays and positrons generate signals with the same waveform within a scintillator, making it difficult to distinguish between gamma rays and positrons within a scintillator using only the PSD.

Meanwhile, in the case of a dual scintillator structure detector with two layers of different scintillators, the difference in the range between positrons and gamma rays can be used to distinguish between positrons and gamma rays. Since positrons have a short range, most positrons incident on the dual scintillator structure detector accumulate energy in the top-layer scintillator and then annihilate with electrons, generating two 511 keV photons in opposite directions. One of the generated 511 keV photons deposits energy in the bottom-layer scintillator. Consequently, when a positron is detected, a coincidence signal is detected, which is a combination of the signals generated from both scintillators (see FIG. 3). Since gamma rays have a long range, most of them deposit energy in the bottom-layer scintillator, and some gamma rays deposit energy in the top-layer scintillator. Therefore, gamma rays deposit energy in only one of the two scintillator layers.

Since the two scintillators that make up the dual scintillator structure detector have different scintillation characteristics, they generate different types of signals depending on the type of scintillator that generated the signal. Therefore, by applying the PSD technique to the acquired signal, it is possible to distinguish between positrons and gamma rays based on the shape of the pulse.

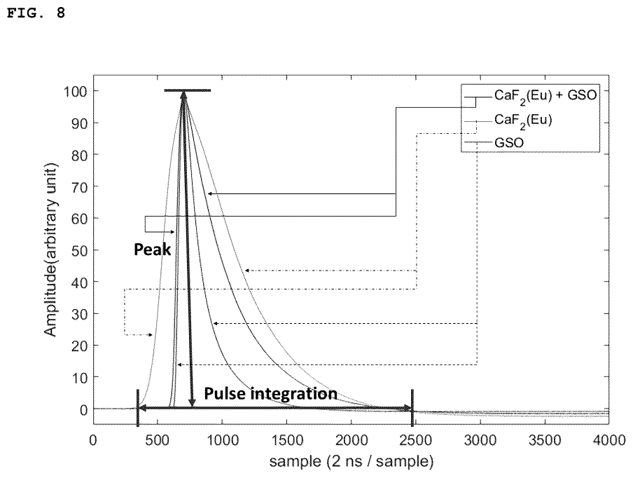

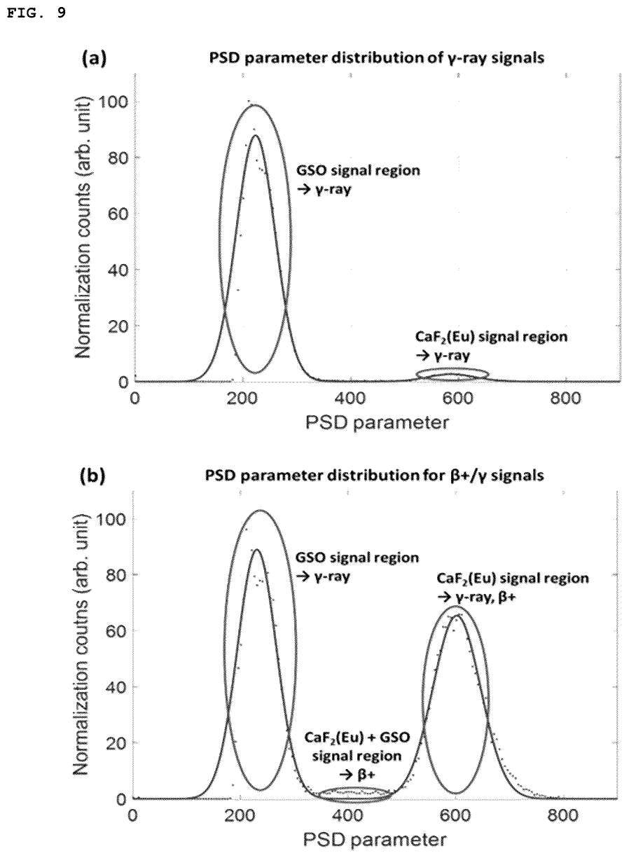

The dual scintillator structure detector used in this Experimental Example 1 consists of a CaF₂(Eu) scintillator and a GSO scintillator. CaF₂(Eu) has a low density, is relatively insensitive to gamma rays, and thus has a long decay signal. The GSO scintillator has a high density, thus is suitable for gamma-ray detection, and has a shorter decay signal than CaF₂(Eu). FIG. 8 is a graph showing the signals of the CaF₂(Eu)/GSO dual scintillator according to Experimental Example 1. The signal generated from each scintillator and the coincidence signal, which is a combination of the signals generated simultaneously from both scintillators, are shown in FIG. 8. The detailed characteristics of each scintillator are listed in Table 1 below.

TABLE 1

| | Light yield | Density | Decay time | Size |
|---|---|---|---|---|
| Scintillator | (photons/MeV) | (g/cm3) | (ns) | (mm³) |
| CaF₂(Eu) | 19,000 | 3.18 | 940 | 3 × 3 × 0.5 |
| GSO | 12,500 | 6.7 | 50 | 3 × 3 × 5 |

Since the decay time versus light output of CaF₂(Eu) is longer than that of GSO, the PSD parameter was defined as in Equation 2 below using the pulse integration and peak of the measured signal.

$$PSD parameter = \frac{pulse intergration}{peak} \qquad [ Equation 2 ]$$

Since CaF₂(Eu) having a longer decay time versus light output has a greater pulse integration versus peak, the signal generated from CaF₂(Eu) has a larger PSD parameter value than the signal generated from GSO.

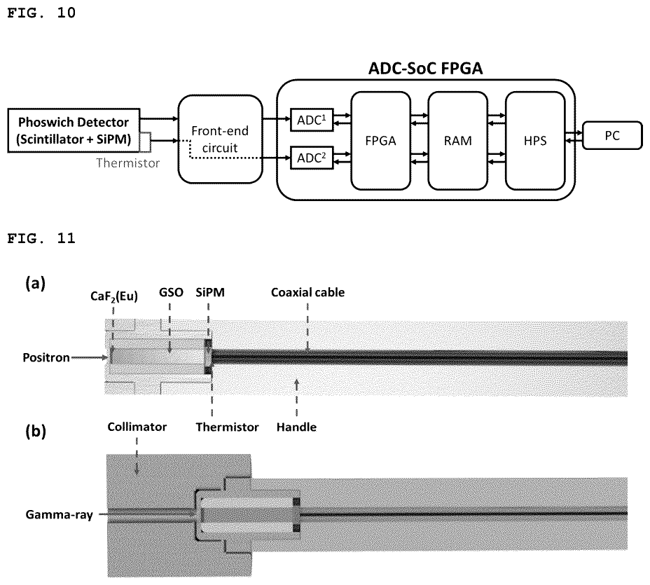

To determine the range of PSD parameter values for distinguishing between positrons and gamma-rays, a ²²Na gamma-ray source and a ²²Na positron source were measured with the dual scintillator structure detector, and the PSD parameters were calculated for each signal to obtain the PSD parameter distribution. FIG. 9 is graphs showing the distributions of PSD parameters for gamma-ray signals (a) and positron/gamma-ray signals (b) of the CaF₂(Eu)/GSO dual scintillator according to Experimental Example 1. FIG. 9(a) shows the PSD parameter distribution of the signals acquired with the ²²Na gamma-ray source, and it can be confirmed that most of the gamma-rays were detected in the GSO scintillator (GSO signal region). On the other hand, FIG. 9(b) shows the PSD parameter distribution of the signals acquired with the ²²Na positron source, and shows that gamma-rays generated by positrons were also detected, indicating the PSD parameter distribution of signals in which the signals of positrons and gamma-rays are mixed. Since the coincidence signal of positrons and gamma rays generated by positron annihilation has a PSD parameter corresponding to the CaF₂(Eu)+GSO signal region, signals having the PSD parameter value of this region can be identified as positrons. The range of the CaF₂(Eu)+GSO signal region was determined by considering both the rate of signals identified as positrons (sensitivity) when measuring the positron source and the rate of signals incorrectly identified as positrons (error rate) when measuring the gamma-ray source. As the PSD parameter range for identification as positrons increased, the sensitivity increased, but the error rate also increased. Conversely, as the range decreased, the error rate decreased, but the sensitivity also decreased. In this experiment, the PSD parameter range was set to have an error rate of 0.5% or less to suppress the influence of gamma rays coming from the background. The set PSD parameter range was 350 to 470, and the sensitivity and error rate for positrons in this range were 1.60% and 0.47%, respectively.

### 1.3 Positron/Gamma-Ray Dual Detection System

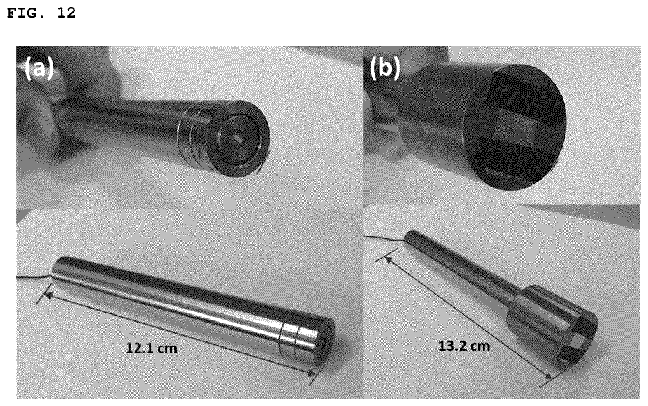

FIG. 10 is a diagram showing the configuration of a positron/gamma-ray dual detection system according to Experimental Example 1. With reference thereto, the positron/gamma-ray dual detection system consisted of a probe for detecting positrons and gamma rays, a front-end circuit for preprocessing signals, and an ADC-SoC FPGA-based digital circuit for digital signal processing (DSP).

The output signal of the positron/gamma-ray dual detection probe is transmitted to the front-end circuit, and this signal is amplified in the front-end circuit and then transmitted to the ADC-SoC FPGA. The PSD parameter of the digitized signal in the ADC-SoC FPGA is calculated using the PSD algorithm, and the signal is identified as a positron or gamma-ray based on the PSD parameter value.

### 1.3.1 Positron/Gamma-Ray Dual Detection Probe

FIG. 11 schematically illustrates a probe of the positron/gamma-ray dual detection system according to Experimental Example 1, and FIG. 12 is photographs showing the gamma-ray mode (a) and the positron mode (b) of the probe shown in FIG. 11.

As illustrated in FIG. 11, the positron/gamma-ray dual detection probe consists of a dual scintillator structure detector, a thermistor for temperature measurement, a collimator, and a handle.

The dual scintillator structure detector is composed of a stack of CaF₂(Eu) and GSO and includes 3×3 mm² SiPM (S13360-3050PE, HAMAMATSU, Japan). The thermistor is disposed close to the SiPM and measures the temperature to compensate for the gain of the SiPM according to the temperature change. The SiPM and the thermistor are connected to the front-end circuit via a coaxial cable (1.1 mm diameter and 3 m length).

The positron/gamma-ray dual detection probe may be used as a high-energy gamma-ray probe or a positron probe, depending on whether a collimator is mounted. In order for the dual detection probe to be used as a high-energy gamma-ray probe, it is required to mount a collimator for shielding background gamma-rays (gamma-ray mode, see FIG. 11(b)). The collimator is manufactured by processing tungsten, is 31 mm in diameter, has a shielding rate of 95%, and blocks 511 keV gamma-rays incident from the side. If the positron/gamma-ray dual detection probe is used without a collimator, it can function as a positron probe (positron mode, see FIG. 11(a)).

Referring to the photograph of the positron/gamma-ray dual detection probe in FIG. 12, the diameter and length of the fabricated probe are 1.5 cm and 12.1 cm, respectively, for the positron mode, and are 3.1 cm and 13.2 cm, respectively, for the gamma-ray mode.

### 1.3.2 Front End and Digital Circuits

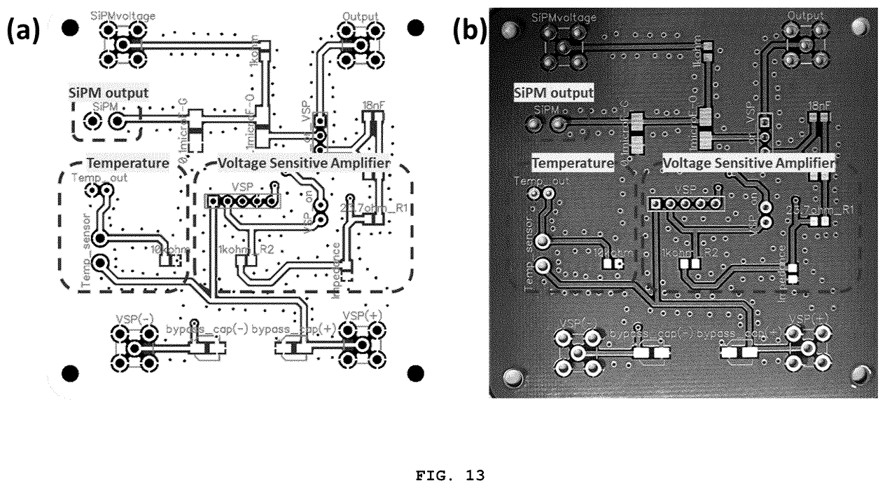

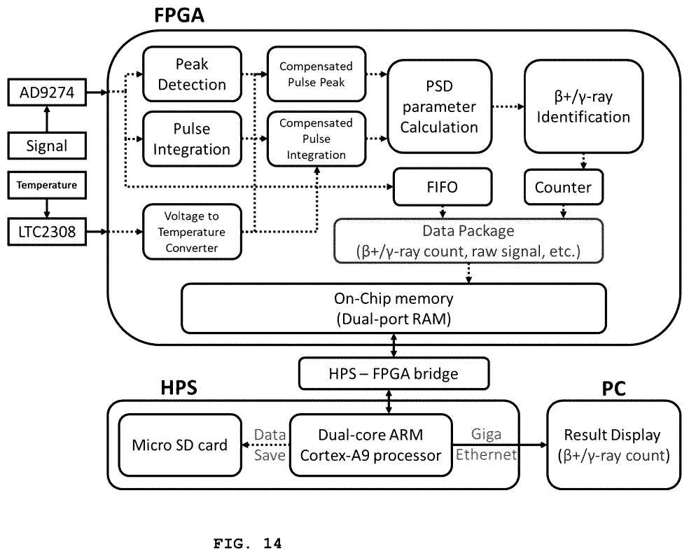

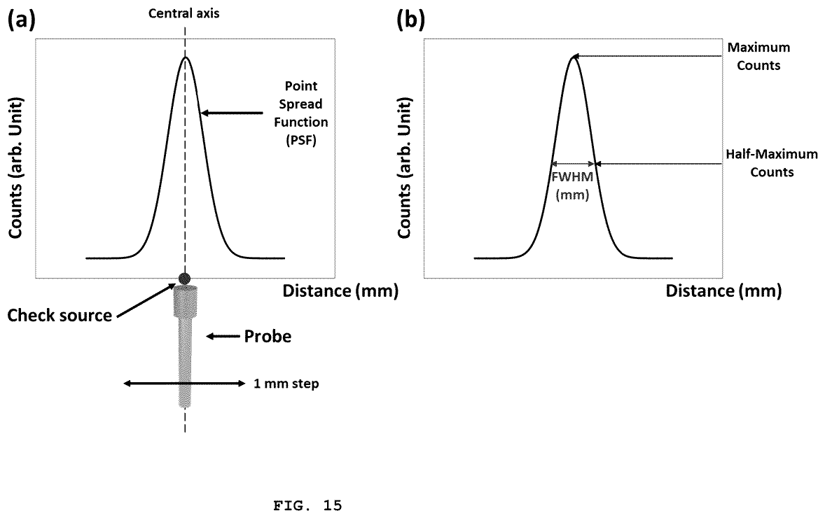

FIG. 13 shows the PCB and PCB layout of the front-end circuit shown in FIG. 10, and FIG. 14 is a flow block diagram of the digital circuit shown in FIG. 10.

Referring to FIG. 13, the output signal of SiPM is amplified by the inverting amplifier in the front-end circuit and then transmitted to the high-speed ADC of the FPGA. Since the resistance value of the thermistor changes with temperature changes, the voltage of the thermistor is transmitted to the low-speed ADC of the FPGA through voltage distribution.

The signal amplified in the inverting amplifier of the front-end circuit and the voltage of the thermistor are transmitted to the high-speed ADC (AD9274, 14-bit, 150 MS/s) and low-speed ADC (LTC2308, 12-bit, 500 kS/s, 8-channel) of the ADC SoC FPGA, respectively.

As shown in FIG. 14, the digital circuit part of this system was designed using an ADC-SoC FPGA. First, the signal transmitted to the high-speed ADC is digitized, and then the peak and pulse integration of the digital signal are calculated.

The thermistor voltage transmitted to the low-speed ADC is converted into temperature using a pre-created voltage-temperature lookup table, and the values of peak and pulse integration are compensated according to the measured temperature. The PSD parameter is calculated using the compensated peak and compensated pulse integration, and the measured signal is identified as a positron or gamma ray depending on the calculated PSD parameter. The counts of positrons and gamma rays are increased according to the identified results, and the counts are packaged together with the original signal and then recorded in the on-chip memory. After the HPS reads the original signal and count data recorded in the on-chip memory, some of the data are saved in a micro SD card, and information on the counts of positrons and gamma rays is transmitted to a PC via gigabit Ethernet. The counts of positrons and gamma rays transmitted to the PC are displayed on the screen in real time.

### 1.4 Performance Test of Positron/Gamma-Ray Dual Detector Probe

To evaluate the performance of the developed positron/gamma-ray dual detection probe, an in vitro test using a check source and an in vivo test using rabbits with skin cancer were performed.

### 4.1 In Vitro Performance Test

The important performance parameters for portable radiation detectors are spatial resolution and sensitivity. Spatial resolution is an indicator of the minimum distance between radiation sources that the probe can distinguish, and reflects the ability of the detector to accurately determine the location of the radiation source. FIG. 15 illustrates an experiment for measuring the performance of the gamma-ray mode and positron mode of the probe, and as shown therein, the detected count rate as a function of the lateral distance from the center of the probe can generate a point spread function (PSF), and the spatial resolution of the probe can be expressed as the FWHM of the PSF.

Another important performance indicator of the probe is sensitivity. However, sensitivity can be somewhat ambiguous, as it is greatly affected by the locations of the source and the detector, as well as the appearance of the detector. In addition, there are various types of sensitivity, such as geometric sensitivity, intrinsic sensitivity, and overall sensitivity, but in this performance evaluation experiment, the overall sensitivity was defined as the sensitivity of the probe and evaluated, and the sensitivity is expressed as the ratio of detected counts per unit activity.

The performance of the positron/gamma-ray dual detection probe was evaluated in each of gamma-ray mode and positron mode. For the gamma-ray mode, spatial resolution and sensitivity were evaluated, and for the positron mode, spatial resolution, sensitivity, and the ratio of false positron detection, so-called error rate, were evaluated. In addition, the spatial resolution and sensitivity of the gamma probe from Crystal Photonics were evaluated for performance comparison with the gamma-ray mode and high-energy probe. The specifications of the portable radiation detection probes used in the experiment are shown in Table 2 below.

TABLE 2

| | Dual detection probe | | | | | |
|---|---|---|---|---|---|---|
| | Positron | | Gamma | | Commercial gamma probe | |
| Specifications | mode | | mode | | (Crystal photonics) | |
| Handle length | 121 | mm | 132 | mm | 220 | mm |
| Cable length | 3 | m | 3 | m | 3 | m |
| Diameter - | 15 | mm | 15 | mm | 20 | mm |
| hand piece | | | | | | |
| Diameter - | 15 | mm | 31 | mm | 15 | mm |

tip

In order to obtain spatial resolution in this experiment, the probe (or collimator in the case of gamma-ray mode) and the source were brought into close contact, and in this state, the counts were measured while moving the probe sideways by 1 mm each time. Measurements were made for 2 minutes at each step, and the spatial resolution was obtained by generating a PSF using the average CPS (see FIG. 15). The check source used in this experiment is a ²²Na gamma-ray source for the gamma-ray mode and a ²²Na positron source for the positron mode. The sensitivity was measured in a state in which the probe and the source were brought into close contact.

### 1.4.2 In Vivo Performance Test

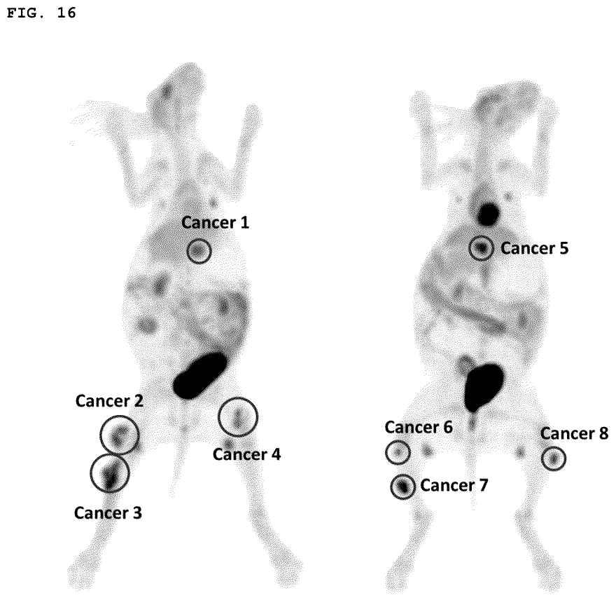

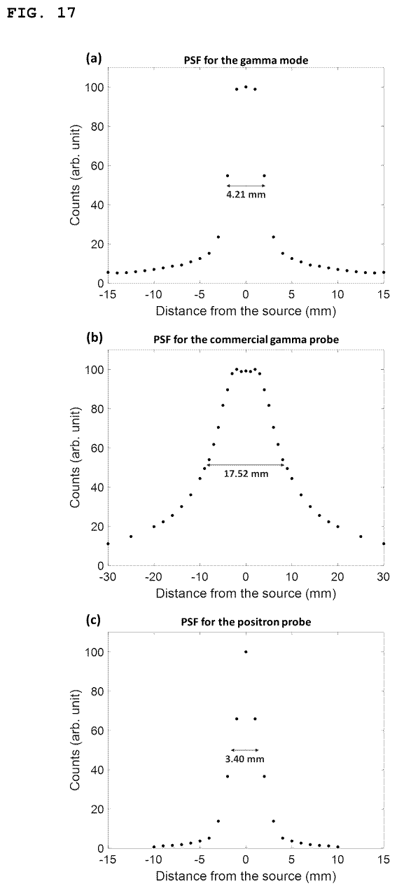

An in vivo test was performed on rabbit skin cancer in order to evaluate the cancer detection performance of the positron/gamma-ray dual detection probe. The most important role of a portable radiation detection probe targeting metabolic targeted resections during surgery is to accurately localize and identify a tumor. Accurate localization of a tumor during surgery can reduce the occurrence of residual cancer after cancer resection, and accurate identification of the tumor can prevent excessive resection of normal tissue during cancer resection surgery, thereby reducing postoperative complications. Accordingly, in this experiment, attempts were made to detect a total of eight skin cancers in two rabbits each having four skin cancers, in order to evaluate the cancer detection performance of the positron/gamma-ray dual detection probe. To evaluate the cancer detection performance, ¹⁸F-FDG was injected into the rabbits, and PET/CT images were taken 30 minutes later, and the images are shown in FIG. 16. FIG. 16 shows PET/CT images of skin cancer in the rabbits used in an animal experiment according to Experimental Example 1. After PET/CT imaging, cancer detection performance evaluation was performed. For the first rabbit, cancer detection performance evaluation was performed approximately 1 hour after FDG injection, and for the second rabbit, cancer detection performance evaluation was performed approximately 2 hours after FDG injection. In cancer detection performance evaluation, in the case of the gamma-ray mode of the positron/gamma-ray dual detection probe, cancer detection was attempted by measuring gamma rays externally without resecting the rabbit skin. In the case of the positron mode, cancer detection was attempted by measuring positrons after excising the skin and opening the cancer. The tumor identification was determined based on the TBR, and the tumor was identified as cancer if the TBR of the tumor was 1.5 or higher. As the background for calculating the TBR, tissue at a distance of 5 cm or less from the tumor was selected.

### 1.5 Results

### 1.5.1 Results of In Vitro Performance Test

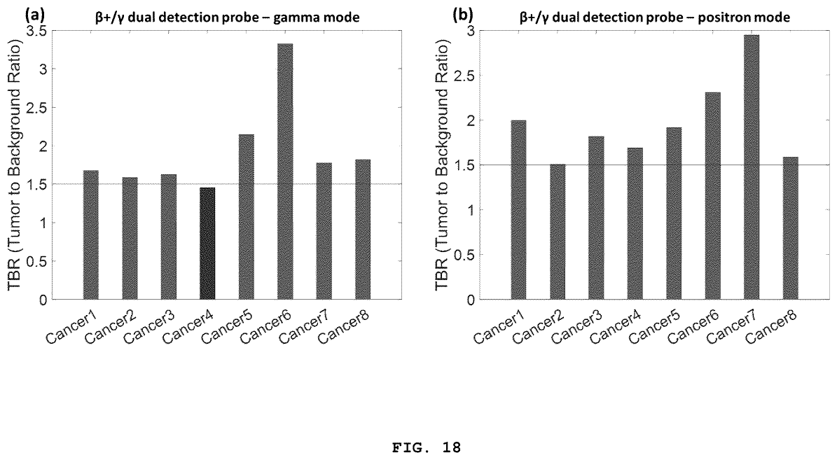

FIG. 17 shows PSF (Point Spread Function) graphs of the positron/gamma-ray dual detection probe and the commercial probe according to Experimental Example 1.

Referring to FIG. 17, the spatial resolution of the positron/gamma-ray dual detection probe was 4.21 mm in the gamma-ray mode, and that of the commercial gamma probe was 17.52 mm. In the positron mode, a spatial resolution of 3.40 mm, which is close to 3 mm, was obtained, because the diameter of the probe tip hole was 3×3 mm² and the active diameter of the ²²Na positron source was 3 mm.

The sensitivity was measured in a state in which the probe and the source were brought into close contact. The sensitivity to gamma rays in the gamma-ray mode of the positron/gamma-ray dual detection probe was 2.16 CPS/μCi, and the commercial probe showed a sensitivity of 9.32 CPS/μCi. The sensitivity to positrons in the positron mode was 204.40 CPS/μCi, and when the tip hole of the probe was blocked with aluminum foil to block light, the sensitivity decreased to 79.58 CPS/μCi.

The error rate was defined as the ratio of gamma-rays incorrectly measured as positrons to the total measured signal when measuring the gamma-ray source in the positron mode. When the error rate was measured using a ²²Na gamma-ray source, the error rate was 0.48%.

### 1.5.2 Results of In Vivo Performance Test

FIG. 18 shows TBR values measured according to the gamma-ray mode (a) and positron mode (b) of the positron/gamma-ray dual detection probe in the animal experiment according to Experimental Example 1, and shows the results of evaluating the cancer detection performance of the positron/gamma-ray detection probe in vivo.

In the gamma-ray mode, 7 out of 8 tumors were identified as cancer, and in the positron mode, all 8 tumors were identified as cancer. Since the heart has a high FDG uptake, cancer 5 around the heart was not identified as cancer in the gamma-ray mode due to the shine-through effect caused by the high background. In the positron mode, which is less affected by the shine-through effect than in the gamma-ray mode, all tumors, including cancer 5, were successfully identified as cancer. In the gamma-ray mode, the average gamma-ray count was 32.68 in the tumor, and 17.37 in the background, and in the positron mode, the average positron count was 11.11 in the tumor, and 5.81 in the background.

### 1.6 Conclusion

In this experimental example, the present inventors fabricated the positron/gamma-ray dual detection probe system and demonstrated that it is possible to successfully perform metabolic targeted resections during surgery by using the dual detection probe system. With the proposed probe, surgeons are expected to be able to accurately localize not only primary tumors but also small residual cancers before resection of the lesion site by using only one probe, thereby completely resecting the cancer.

In the in vitro evaluation of the positron/gamma-ray dual detection probe system, the spatial resolution in the gamma-ray mode was 3.91 mm and the sensitivity was 2.16 cps/μCi, and the spatial resolution in the positron mode was 3.27 mm and the sensitivity was 204.40 cps/μCi. Meanwhile, the spatial resolution and sensitivity of the commercial gamma probe were 15.62 mm and 9.32 cps/μCi, respectively. It is analyzed that the spatial resolution and sensitivity of the commercial gamma probe were greater because the commercial gamma probe used a relatively thinner collimator than the gamma-ray mode of the positron/gamma-ray dual detection probe.

In the in vivo evaluation of the positron/gamma-ray dual detection probe system, the average CPS in the gamma-ray mode of the positron/gamma-ray dual detection probe was 32.68 in the tumor and 17.37 in the background. The average CPS in the positron mode was 11.11 in the tumor and 5.81 in the background. In the in vitro test using the check source, the sensitivity of the positron mode was higher than that of the gamma-ray mode, but the in vivo results of an in vivo test using animals, the opposite results were obtained. This is because in the case of the gamma-ray mode, the penetration depth of gamma rays is large and the FOV is wide, and thus gamma-rays incident over a wide range are detected, but in the positron mode, only positrons emitted from the area where the positron/gamma-ray dual detection probe is in close contact are detected. However, in the cancer detection experiment, the range of TBR obtained for each tumor by the positron/gamma-ray dual detection probe was 1.46 to 3.33 in the gamma-ray mode and 1.51 to 2.95 in the positron mode. In conclusion, the positron/gamma-ray dual detection probe successfully identified 7 out of 8 cancers as cancer in the gamma-ray mode, and successfully identified all 8 tumors as cancer in the positron mode. Therefore, it can be seen that the positron/gamma-ray dual detection probe system according to the present invention may be used for real-time cancer identification during surgery. The positron mode may exhibit lower sensitivity than expected when detecting positrons, which may result in a long detection time for detecting small tumor lesions. Therefore, the positron/gamma-ray dual detection probe to which a positron algorithm with reasonably high sensitivity and accuracy is applied may be the best choice to overcome these limitations.

### 2. New Cancer Detection Index Using Positron/Gamma-Ray Dual Detection Probe

### 2.1 Introduction

For many cancers, the risk of recurrence remains high despite the assumption of complete resection at the time of initial surgical management. This means that occult cancers may remain undetected at the time of surgery and may not be completely resected by surgical approaches. In this regard, the introduction of ¹⁸F-FDG positron emission tomography/computed tomography (PET/CT), a functional imaging technique, has significantly improved the detection of preoperative occult cancer and tumor localization. Although the specificity of ¹⁸F-FDG to some malignant tumors is limited, several studies have reported the clinical value of ¹⁸F-FDG-PET/CT in the diagnosis of various primary and metastatic cancers, including occult lesions with clinically negative or equivocal findings on imaging. Nevertheless, preoperative PET/CT images cannot provide real-time information for intraoperative tumor localization and immediate verification of complete tumor resection, and thus it is often difficult to localize and completely resect tumors, found on preoperative PET/CT images, during surgery. Therefore, probe-guided surgery that uses a portable radiation detection probe to localize the tumor location in real time during surgery and verify tumor resection may be one solution. In probe-guided surgery, a portable radiation detection probe helps the surgeon to selectively resect malignant tissue without leaving any residual cancer during surgery. In particular, a positron probe, one of portable radiation detection probes, can directly detect positrons emitted from positron-emitting radiopharmaceuticals that accumulate in malignant tumors thereby localizing malignant tumors. Unlike gamma rays, positrons travel only a few millimeters in tissue, and thus if the positron probe is placed close to the tumor, the location of the malignant tumor can be accurately identified.

In Experimental Example 1, the positron/gamma-ray dual detection probe system that can be used for metabolic targeted resections during surgery was developed, and malignant tumors were successfully detected in the in vivo test conducted using the medium-sized animal. However, there are still several issues to consider in terms of sensitivity to positrons and background selection for calculating TBR. Portable radiation detection probes targeting ¹⁸F-FDG detect cancer by exploiting the Warburg effect, which causes high accumulation of ¹⁸F-FDG in many cancers compared to background tissue, often exceeding 10:1. Portable radiation detection probes based on this property identify cancer by comparing radiation counts in the tumor with counts in the background. TBR is the ratio of the signal counts measured in the tumor to the signal counts measured in the background, and empirically, a tumor with a TBR value of 1.5 or greater is identified as a malignant tumor. TBR, which uses radiation counts as a parameter for cancer detection, has successfully identified malignant tumors in many studies conducted using radiation detection probes, but calculating the TBR value requires not only the radiation counts in the tumor but also the counts in the background. This means that the TBR value can change not only depending on the radiation counts in the tumor but also depending on the counts in the selected background. Therefore, the results of identification of a malignant tumor can vary depending on the location of the selected background even for the same tumor. In fact, since ¹⁸F-FDG accumulates a lot in specific tissues such as the kidney, heart, and liver, lesions existing around these specific tissues may not be easy to distinguish from the background.

One way to accurately identify malignant tumors with a radiation detection probe is careful background selection. However, there is ambiguity in the selection of the background area. Some studies have offered guidelines for background selection, but the guidelines vary from study to study. For portable positron probes, strict criteria for positron identification can be applied to suppress background gamma-ray counts, but this can act as a factor that reduces the sensitivity of the positron probe to positrons. Due to the direct positive correlation between the counts in the tumor of the positron probe and the tumor size, positron probes with low sensitivity have difficulty in detecting small tumors, which makes it difficult to provide immediate information on the complete resection of the tumor during surgery. In this way, the results of cancer identification can vary depending on the background selection rather than the tumor. Therefore, in order to provide surgeons with immediate information on the tumor location and complete resection of the tumor during surgery using a portable positron probe, cancer detection parameters that are not affected by the background location and do not reduce the sensitivity of the positron probe are required.

In this experimental example, the present inventors propose a new parameter for cancer detection, PGR (positron to gamma ratio), which can be used in the positron mode of the positron/gamma-ray detection probe. The proposed PGR shows a constant value regardless of the location of the selected background, and thus it can detect cancer more accurately than cancer detection using positron counts, and it is expected to show excellent performance in detecting small-sized tumors. Accordingly, the present inventors designed a study to verify the ability of the proposed new parameter for cancer detection to localize tumors and detect small-sized tumors. First, in an in vitro test using a phantom composed of a gamma-ray source and a positron-ray source, the present inventors confirmed that PGR showed a constant value regardless of the location of the background. Second, in an in vivo test using medium-sized animals, the present inventors attempted to identify tumors and small tumors present in tissues with high ¹⁸F-FDG uptake, such as kidney, heart, and liver, using PGR. The overall goal of this experiment is to accurately identify malignant tumors during surgery regardless of their location and size using the developed positron/gamma-ray detection probe and the new parameter for cancer detection, PGR, thereby assisting in complete resection of malignant tumors.

### 2.2 Cancer Detection Index

Portable positron probes identify malignant tumors by using the ratio of positron counts in tumor and background (TBR). It is known that positron probes are able to accurately localize malignant tumors because they directly detect positrons that travel only a very short distance within tissue, and are not affected by the shine-through effect caused by high background positrons. However, since many gamma-rays generated by electron-positron annihilation and bremsstrahlung within the tissue can be misidentified as positrons within the positron probe, suppression of background gamma-rays is necessary. Therefore, in order to suppress background gamma-rays, the PSD technique was applied to the positron/gamma-ray dual detection probe in Experimental Example 1 above to distinguish and detect positrons and gamma-rays. Even if positrons are identified using the PSD technique, some gamma rays can still be misidentified as positrons (error rate), but it is possible to maintain a low error rate of 0.5% by applying a strict PSD technique. However, due to the absence of a collimator to block background gamma rays, as well as the strong penetrability of gamma rays and the very weak penetrability of positrons, the number of gamma rays reaching the positron probe close to a tumor may be much higher than that of positrons. For this reason, despite the low error rate, higher positron counts may be measured around organs that emit a lot of background gamma rays than in tumors, making it often difficult to identify malignant tumors based on positron counts. Therefore, for consistent identification of malignant tumors, a cancer detection index that has a constant value in the background regardless of the locations of the tumor and the background and shows a higher value in the malignant tumor than in the background is required. Accordingly, the present inventors propose a new cancer detection index called PGR, taking into account the fact that the error rate of the positron/gamma-ray dual detection probe always shows a constant value. Since the positron/gamma-ray dual detection probe can detect positrons and gamma-rays simultaneously, PGR was defined as Equation 3 below by dividing the simultaneously measured positron count by the measured gamma-ray count.

$$PGR = {Counts}_{β} / {Counts}_{γ - ray} \qquad [ Equation 3 ]$$

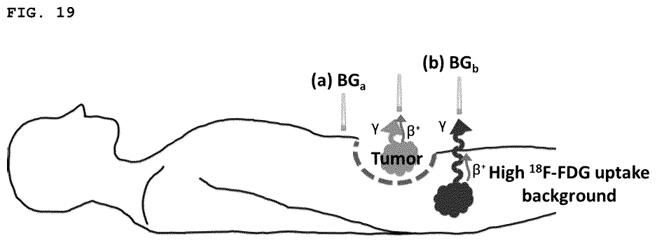

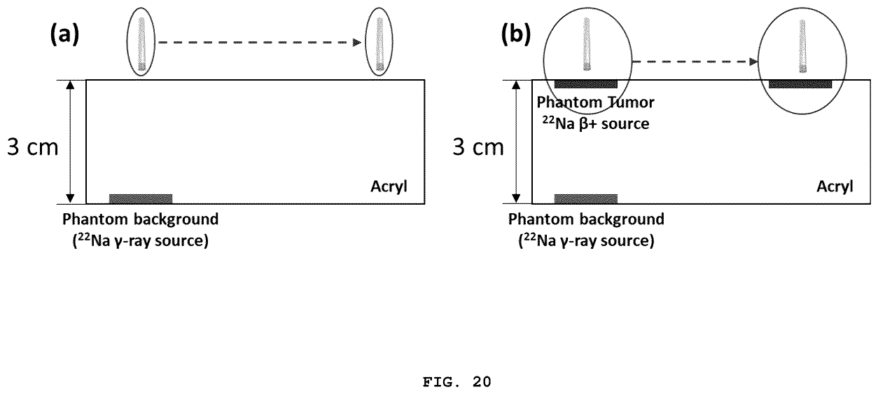

Since the rate of false positrons misidentified as gamma rays is constant, the PGR in the background has a constant value, and thus the PGR-based malignant tumor identification method can identify tumors more consistently than a conventional method of identifying a malignant tumor based on the positron count. FIG. 19 illustrates a conventional positron count-based malignant tumor detection method. For example, in a background with a high ¹⁸F-FDG uptake as shown in (b) of FIG. 19, more gamma rays are emitted, resulting in a greater number of misidentified positrons than that in another background shown in (a) of FIG. 19. Therefore, the TBR values may differ even for the same tumor depending on the selected background, and thus consistent tumor identification is difficult. On the other hand, in the backgrounds shown in (a) and (b) of FIG. 19, the PGR values are almost the same, and thus consistent tumor identification is possible.

To compare the traditional cancer detection method based on the radiation count with the cancer detection method based on PGR, a newly proposed index for cancer detection, the positron/gamma-ray dual detection probe developed in Experimental Example 1 was used.

### 2.3 Cancer Detection Tests

### 2.3.1 In Vitro Test

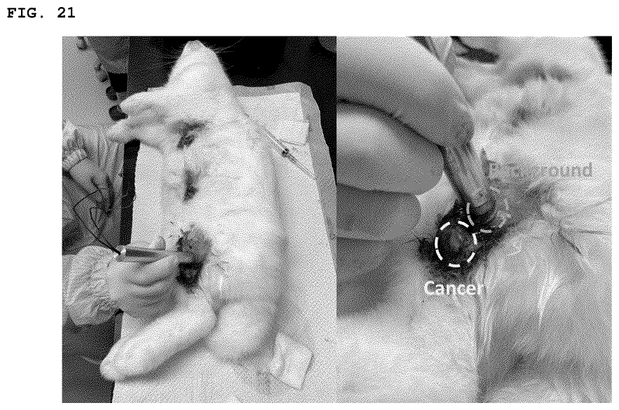

FIG. 20 illustrates an in vitro test according to Experimental Example 2. The performance of positron count-based and PGR-based cancer detection methods was compared by measuring a ²²Na gamma-ray source and a positron source using the positron/gamma-ray dual detection probe. First, as shown in FIG. 20(b), in order to confirm the changes in the positron count and PGR value measured using the positron/gamma-ray dual detection probe depending on the position of the background, the gamma-ray source and the positron/gamma-ray dual detection probe were placed coaxially at a distance of 3 cm and the gamma-ray source was fixed, and in this state, the positron count and PGR value were measured while laterally moving the probe up to 26 mm. Since the probe and the gamma-ray source were blocked from each other by an acryl plate, only gamma rays could reach the detector. Then, as shown in FIG. 20(b), in a state in which the ²²Na gamma-ray source was fixed, the ²²Na positron source was placed at a distance of 3 cm along the same axis, and the positron count and the PGR value were measured while the positron source and the probe were moved laterally while in close contact.

The positron count and PGR value obtained in (a) and (b) of FIG. 20 were assumed as measurement values for the background and the tumor, respectively. In the case of the conventional positron count-based cancer detection method, 1.5 times the positron count obtained in (a) of FIG. 20 was set as the criterion for identifying a malignant tumor for each lateral distance between the probe and the gamma-ray source (TBR>1.5). In the PGR-based cancer detection method, the mean and standard deviation of the PGR values obtained in (a) of FIG. 20 were used as criteria for identifying a malignant tumor. In this experiment, the mean PGR value plus three times the standard deviation was used as the criteria for identifying a malignant tumor. The cancer detection ability of each parameter for cancer detection was evaluated using the malignant tumor identification criteria of each parameter for cancer detection.

### 2.3.2 In Vivo Test

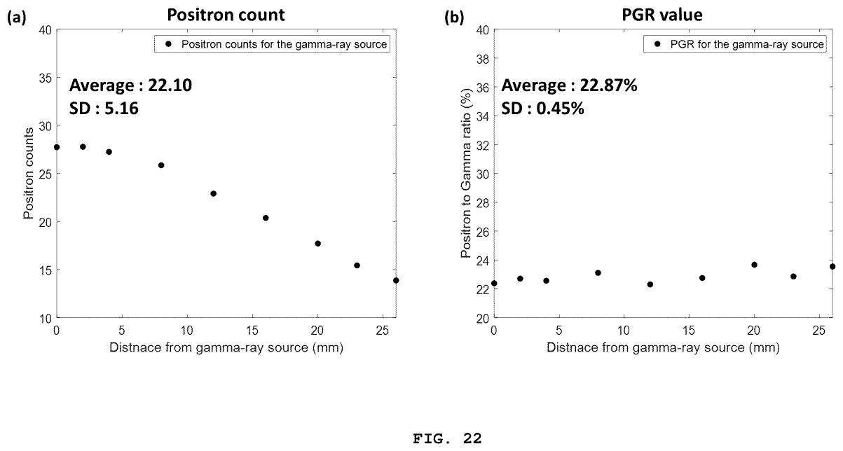

FIG. 21 illustrates an in vivo test according to Experimental Example 2. Referring thereto, an in vivo test was performed to evaluate the performance of the positron count-based and PGR-based cancer detection methods using the positron/gamma-ray dual detection probe. The experimental subjects were four rabbits in total, two of which each had four skin cancers, and the remaining two rabbits each had five skin cancers. To compare the cancer detection performance of the two indices for cancer detection depending on the location of the tumor, some of the tumors were located in the heart and abdomen, where the background with high ¹⁸F-FDG uptake was distributed. The remaining tumors were located on the legs with relatively little background. In vivo tests were performed on four rabbits (two rabbits per day) on different days, and were performed on a total of 18 skin cancers. Each rabbit was injected with ¹⁸F-FDG, PET/CT images were taken 30 minutes after injection, and in vivo tests were performed after the imaging. In vivo tests were started approximately 1 hour after FDG injection for the first rabbit, and approximately 2 hours after FDG injection for the second rabbit.

For all the tumors, the skin was incised to expose the tumor. In this state, two areas at a distance of 1 cm or less from the same tissue as the tumor were selected as a background, and the positron counts and PGR of the tumor and background were measured, and malignant tumors were identified based on the measurements. For positron counts, a tumor was identified as malignant if its TBR was 1.5 or higher, and for PGR, a tumor was identified as malignant if the PGR value measured in the tumor was greater than the mean of the PGR values measured in the background plus three times the standard deviation. In addition, to evaluate the cancer detection ability depending on the tumor size, the changes in positron count and PGR value depending on the tumor size were measured while resecting the tumor in vivo. Based on the results of malignant tumor identification, the cancer detection ability of the cancer detection parameters was compared.

### 2.4 Results

### 2.4.1 In Vitro Test Results

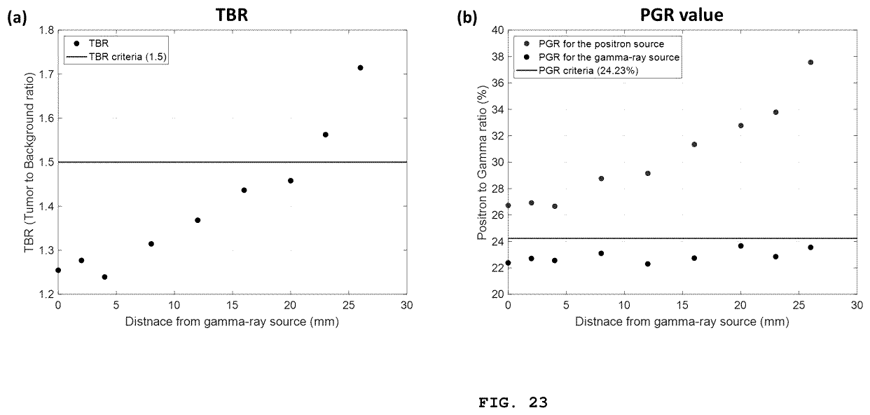

FIG. 22 is graphs showing the changes in positron count (a) and PGR value (b) as a function of the distance between the ²²Na gamma-ray source and the positron/gamma-ray dual detection probe in the in vitro test according to Experimental Example 2.

As shown in FIG. 22(a), the positron count of the positron/gamma-ray dual detection probe for the gamma-ray source gradually decreased as the distance from the gamma-ray source increased, and the average positron count was 22.10 and the standard deviation was 5.16. On the other hand, as shown in FIG. 22(b), the PGR value was constant regardless of the distance from the gamma-ray source. The average PGR value was 22.87% and the standard deviation was 0.45%. The relative standard deviations of the positron count and PGR of the positron/gamma-ray dual detection probe with respect to the distance from the gamma-ray source were 23.35% and 1.98%, respectively, which proves that the PGR value measured by the positron/gamma-ray dual detection probe is constant regardless of the distance from the gamma-ray source.

FIG. 23 is graphs showing the changes in positron count (a) and PGR value (b) as a function of the distance between the positron/gamma-ray dual detection probe in close contact with a positron source and the gamma-ray source in the in vitro test.

Referring to FIG. 23, the measured positron count decreased from 34.79 to 23.78 as the distance from the gamma-ray source increased, and the PGR value increased from 26.72% to 37.56%. The cancer discrimination criterion for the positron count was determined to be 1.5, which means that the ratio of the positron count for the positron source to the positron count for the gamma-ray source at the same distance is 1.5. The cancer identification criterion of PGR was 24.23%, which is the average PGR value for the gamma-ray source plus three times the standard deviation. In the positron count-based cancer identification method, the positron source could be identified when the distance between the gamma-ray source and the positron source was 2 cm or more, and in the PGR-based cancer identification method, the positron source could be identified regardless of the distance between the gamma-ray source and the positron source. This demonstrates that the PGR-based index for cancer detection consistently identifies malignant tumors regardless of the locations of the tumor and background.

### 2.4.2 In Vivo Test Results

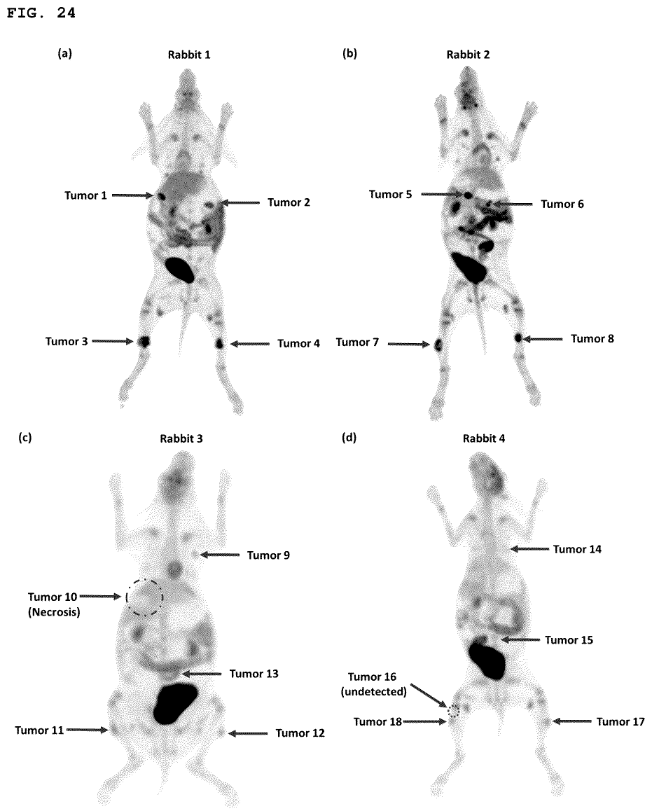

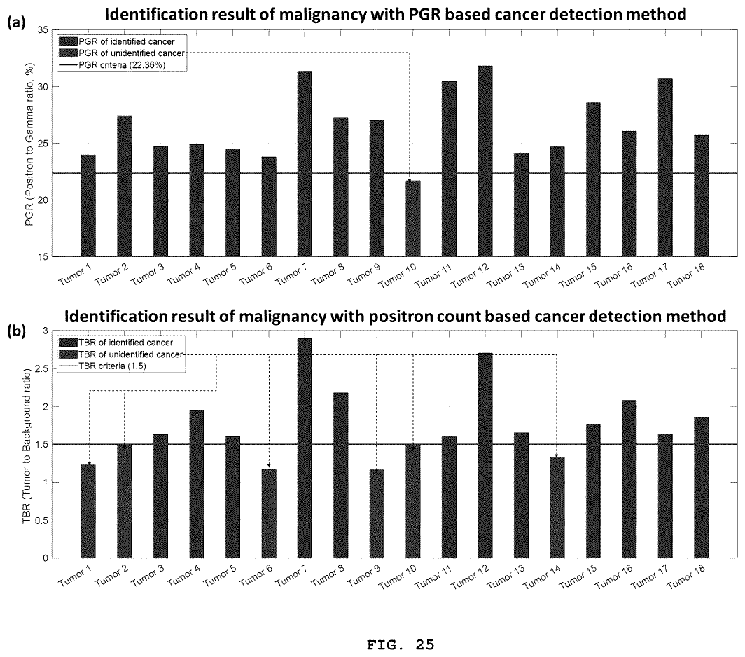

FIG. 24 shows PET/CT images of rabbit tumors taken before the in vivo test according to Experimental Example 2, and FIG. 25 shows the results of the in vivo test according to Experimental Example 2.

Referring to FIG. 24, 16 out of 18 tumors could be found in the PET/CT images taken before the in vivo test. Of the two tumors not found in the PET/CT images, one was a necrotic tumor (Tumor 10), and the remaining tumor (Tumor 16) was small in size (0.4×0.3×0.3 mm³), was located close to another tumor (Tumor 18), and thus was not found in the PET/CT images.

Referring to FIG. 25, in the in vivo test using the positron/gamma-ray dual detection probe, the PGR-based malignant tumor identification method successfully identified 17 out of 18 tumors, excluding a necrotic tumor (Tumor 10).

Table 3 below shows PGR values for tumor and background.

TABLE 3

| | Positron count | | | PGR | |
|---|---|---|---|---|---|
| Tumor | TBR | tumor | background | tumor | background |
| 1 | 1.17 | 35.69 | 30.46 | 23.97% | 18.91% |
| 2 | 1.44 | 32.22 | 22.34 | 27.43% | 17.83% |
| 3 | 1.57 | 29.50 | 18.77 | 24.70% | 18.70% |
| 4 | 1.71 | 27.69 | 16.24 | 24.89% | 16.90% |
| 5 | 1.36 | 58.76 | 43.07 | 24.45% | 19.39% |
| 6 | 1.06 | 36.91 | 34.71 | 23.79% | 20.04% |
| 7 | 2.78 | 48.11 | 17.33 | 31.29% | 16.62% |
| 8 | 1.86 | 35.08 | 18.91 | 27.26% | 18.34% |
| 9 | 1.13 | 37.91 | 33.69 | 27.01% | 19.58% |
| 10 | 1.27 | 52.13 | 40.98 | 21.70% | 18.68% |
| 11 | 1.44 | 38.53 | 26.70 | 30.46% | 19.78% |
| 12 | 2.52 | 42.98 | 17.08 | 31.81% | 16.43% |
| 13 | 1.42 | 84.60 | 59.77 | 24.15% | 18.14% |
| 14 | 1.29 | 29.52 | 22.97 | 24.69% | 19.63% |
| 15 | 1.66 | 41.03 | 24.78 | 28.57% | 18.48% |
| 16 | 1.98 | 28.46 | 14.35 | 26.06% | 20.61% |
| 17 | 1.60 | 27.82 | 17.41 | 30.67% | 19.98% |
| 18 | 1.69 | 26.46 | 15.61 | 25.70% | 19.91% |

The PGR values measured in the remaining tumors excluding the necrotic tumor varied from 23.79% to 31.29%, and the average PGR value for all the tumors was 26.59±2.85%. The background selected for tumor identification was the same tissue within 1 cm from the tumor, and the PGR values measured for the background varied from 16.43% to 20.61%. The average PGR value for the background was 18.78±1.19%, and the PGR criterion for identifying malignant tumors was 22.36%. The PGR value measured in the necrotic tumor was 21.70%, which was not identified as a malignant tumor. In conclusion, since the deviation of the PGR values measured in the background was small, all cancers except the necrotic tumor were successfully detected using the PGR-based malignant tumor identification method regardless of the tumor location.

In contrast, the conventional TBR-based malignant tumor identification method identified only 12 out of 18 tumors as malignant tumors. The positron counts measured in the tumors varied from 26.46 to 94.60, and the average positron count measured for all the tumors was 39.63±13.98. The background positron counts measured in the same tissue located within 1 cm from the tumor varied from 14.35 to 59.77, and the mean positron count measured for the background was 26.40±11.79. To identify malignant tumors, the TBR was calculated using the positron counts measured for the tumor and background. The TBR criterion for identifying malignant tumors was 1.5, and the TBR measured for 18 tumors varied from 1.17 to 2.90. All six tumors that were not identified as malignant, including one necrotic tumor (Tumor 10), due to the large deviation of the positron count measured in the background, were located in the heart or abdomen.

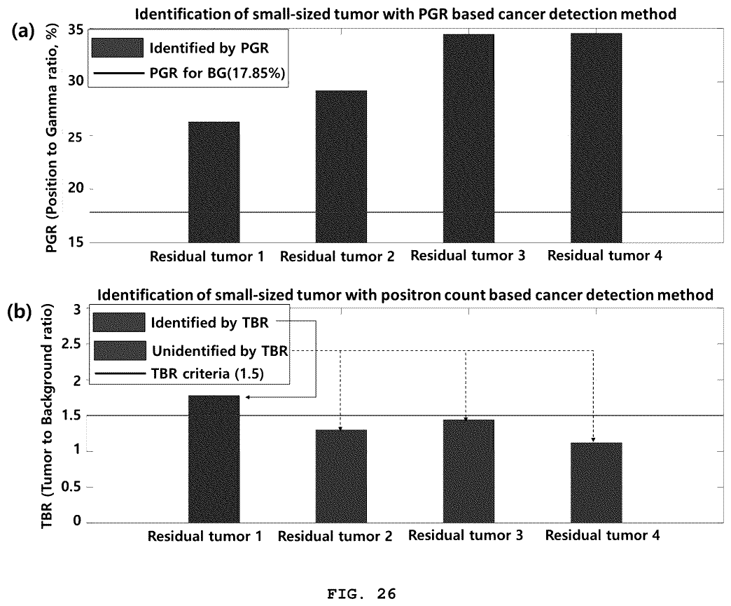

FIG. 26 is graphs showing the positron count and PGR value of residual cancer according to Experimental Example 2. The positron count and PGR value measured in the evaluation of cancer detection ability depending in the tumor size are shown in FIG. 26. The positron count and PGR of residual cancer were measured while resecting Tumor 8 with a size of 18×13×4 mm³ to a size of 5×2.5×3 mm³. The results are summarized in Table 4 below.

TABLE 4

| | | | |
|---|---|---|---|
| Residual | | | |
| Tumor | Size | Positron count | PGR |

| | | | | |
|---|---|---|---|---|
| 1 | 18 × 13 × 7 | mm³ | 43.64 | 26.28% |
| 2 | 18 × 5 × 4 | mm³ | 31.87 | 29.19% |
| 3 | 5 × 5 × 4 | mm³ | 35.15 | 34.44% |
| 4 | 5 × 2.5 × 3 | mm³ | 27.42 | 34.54% |

| | | |
|---|---|---|
| Background | 24.46 | 17.85% |

Referring to FIG. 26 and Table 4 above, the TBR values measured in the tumors decreased as the tumor size decreased. This is because the positron count measured in the tumor decreased as the tumor size decreased. On the other hand, the PGR values measured in the tumors tended to increase as the tumor size decreased. In the case of gamma rays with strong penetrability, all gamma rays generated from the entire tumor are detected, whereas positrons have weak penetrability, and thus only positrons generated from the tumor surface are detected. Therefore, as the tumor size decreases, the number of measured positrons decreases somewhat, but this decrease is relatively small compared to the decrease in measured gamma rays, and thus the PGR value increases rather than decreases. As a result, in the identification of malignant tumors using TBR calculated based on positron counts, small-sized tumors could not be identified, but in the identification of malignant tumors based on PGR, the smaller the tumor size, the easier it was to identify malignant tumors.

### 2.5 Conclusion

In this experiment, the present inventors proposed a new parameter for cancer detection, PGR, which shows a constant value regardless of the location of the selected background. The proposed PGR could detect cancer more accurately than cancer detection using positron counts, and exhibited excellent performance in detecting small-sized tumors.

In the in vitro test conducted to determine the deviation of cancer detection parameters depending on the distance from the background, the positron count increased as the distance from the background (gamma-ray source) decreased, and the average positron count was 22.10±5.16 at a distance of 0 to 26 mm, and the relative standard deviation was 23.35%. On the other hand, the PGR value was relatively constant regardless of the distance from the background (gamma-ray source). The average PGR value measured over the entire distance from the gamma-ray source was 22.87±0.45%, and the relative standard deviation was 1.98%. The PGR value showed a constant value with a small deviation compared to the positron count regardless of the distance from the background emitting a large amount of gamma rays.

In the in vivo tests conducted to evaluate the ability of cancer detection indices to detect tumors in various locations, conventional positron count-based tumor identification failed to identify six of nine malignant tumors located in the heart or abdomen with high ¹⁸F-FDG uptake. On the other hand, the PGR-based malignant tumor identification method successfully identified 17 out of 18 malignant tumors, excluding the necrotic tumor, regardless of the tumor location. In addition, PGR was suitable for detecting small-sized tumors because the measured value increased as the tumor size decreased, unlike positron counts.

Identification of malignant tumors using the positron/gamma-ray dual detection probe and PGR during surgery is expected to enable consistent identification of malignant tumors regardless of the location and size of the tumor, and to help surgeons selectively resect only malignant tissue without leaving residual cancer.

Although the present invention has been described in detail with reference to the specific embodiments, these embodiments are intended to describe the present invention in detail, and the present invention is not limited thereto. It will be apparent to those skilled in the art that modifications and improvements are possible without departing from the technical spirit of the present invention.

All simple modifications or changes of the present invention fall within the scope of the present invention, and the specific protection scope of the present invention will become clear from the appended claims.

### INDUSTRIAL APPLICABILITY

The present invention relates to a nuclear medicine positron/gamma-ray dual detector capable of simultaneously measuring positrons and gamma rays, and a method for providing information for diagnosing a malignant tumor, which is capable of detecting a malignant tumor by establishing a new cancer detection index, which is not dependent on the location of the malignant tumor, by using the dual detector. Thus, the present invention is industrially applicable.

1. A positron/gamma-ray dual detector comprising:
a scintillator unit comprising a first scintillator configured to react with positrons and emit a first scintillation, and a second scintillator disposed adjacent to the first scintillator and configured to react with gamma rays transmitted through the first scintillator and emit a second scintillation;
a photosensor configured to detect any one or more of the first scintillation and the second scintillation and generate and output a detection signal;
a casing configured to accommodate therein the scintillator unit and the photosensor and having an inlet hole formed at one end to allow the positrons or the gamma rays to enter the first scintillator; and
a signal processing unit configured to receive the output detection signal and identify the positrons and gamma rays, which entered through the inlet hole, based on the received detection signal.

2. The positron/gamma-ray dual detector of claim 1, further comprising a collimator which is detachably attached to one end of the casing and has a gamma-ray passage that communicates with the inlet hole of the casing so that the gamma rays emitted in a predetermined direction are incident on the first scintillator.

3. The positron/gamma-ray dual detector of claim 2, wherein one end of the casing is inserted into and screwed to the collimator, and the dual detector further comprises a ring-shaped cover sleeve that is screwed to one end of the casing after the collimator is detached.

4. The positron/gamma-ray dual detector of claim 1, wherein the first scintillator comprises any one or more selected from the group consisting of CaF₂(Eu), plastic, and stilbene.

5. The positron/gamma-ray dual detector of claim 1, wherein the second scintillator comprises any one or more selected from the group consisting of GSO, GPS, LuAG, GAGG, BGO, YAP, and LYSO.

6. The positron/gamma-ray dual detector of claim 1, further comprising a temperature sensor that detects a temperature of the photosensor and generates and outputs temperature information,
wherein the signal processing unit receives the output temperature information and compensates for the detection signal.

7. The positron/gamma-ray dual detector of claim 1, further comprising:
a handle connected to the other end of the casing and formed to be gripped by a user; and
a signal cable that penetrates an inside of the handle and transmits the detection signal to the signal processing unit.

8. The positron/gamma-ray dual detector of claim 1, wherein the signal control unit identifies the positrons and the gamma rays based on a waveform of the detection signal.

9. A method for providing information for diagnosing a malignant tumor, comprising:
step (a) of sequentially placing the positron/gamma-ray dual detector of claim 1 on multiple tissue regions of a subject's body, identifying positrons and gamma-rays emitted from each of the tissue regions, and counting the positrons and the gamma-rays;
step (b) of calculating a cancer index value for each of the tissue regions according to Equation 1 below; and
step (c) of comparing the cancer index value calculated for each of the tissue regions with a predetermined critical value and determining that the tissue region corresponding to the cancer index value exceeding the critical value is a malignant tumor tissue:
$$PGR = \frac{{Counts}_{β}}{{Counts}_{γ - ray}} \qquad [ Equation 1 ]$$
wherein PGR is the cancer index value, Counts_{β} is the positron count, and Counts_{γ-ray} is the gamma-ray count.

10. The method of claim 9, wherein the critical value is an average value of the cancer index values calculated for each of the tissue regions plus n times a standard deviation (where n is a natural number greater than or equal to 1).

11. The method of claim 10, wherein n is 3.
